(19) United States
(12) Patent Application Publication
(10) Pub. No.: US 2007/0233116 A1
(43) Pub. Date: Oct. 4, 2007

(54) BONE FIXATION ASSEMBLY

(76) Inventor: Sven OLERUD, Lanna (SE)

Correspondence Address: DAVID I. ROCHE;BAKER & MCKENZIE LLP, 130 EAST RANDOLPH DRIVE, CHICAGO, IL, 60601, US

(21) Appl. No.: 11/670,504

(22) Filed: Feb. 2, 2007

(30) Foreign Application Priority Data
Mar. 17, 2006 (SE) 0600610-0

(51) Int. Cl. A61F 2/30

(52) U.S. Cl. 606/69

(57) ABSTRACT

The present invention pertains to an arrangement, as well as a method, for fastening and fixing of first element, in the form of an implant, against the second element, in the form of bone tissue, during surgical use of a screw joint, by means of a screw with a conical head with a cone toward the screw tip, said head having an outside thread with a small pitch, which is intended to be screwed into the implant, and a main thread on the stem of the screw, which has greater pitch than the thread of the screw head, and said thread is intended to be screwed into the bone tissue, in which said first element, the implant, has a spherical hole, in which a ductile sleeve is arranged in the form of a sleeve divided into segments, flanges, and has a spherical shape that matches the spherical shape of hole, and said sleeve has a conical threaded inside shape that is complementary to the outer threaded shape of the screw head, in which screw, when tightened, can fasten the implant in any desired angle, relative to the other element, and fix the screw joint in this position.

### TECHNICAL AREA OF THE INVENTION

The present invention concerns an arrangement, for example, during surgical use of a screw joint for fastening and fixing a first element, for example, in the form of an implant or a combination implant, against another element, for example, in the form of bone tissue, by means of a screw containing, on the one hand, a conical head with a cone toward the screw tip, said head having an outside thread with a small pitch, which is intended to be screwed into the first element, for example, the implant; and, on the other hand, a main thread on the screw's stem, which has greater pitch than the pitch of the screw head and is intended to be screwed into the other element, for example, the bone tissue, said screw being fastenable at a desired angle relative to the element and fixed in this position.

### TECHNICAL BACKGROUND OF THE INVENTION

There are many different solutions and methods for fastening elements to each other. The use of traditional screws and an implant plate, for example, when an implant is to be fastened to a bone in orthopedic surgery, has many drawbacks. When the implant plate is fastened to the bone, a pressure relative to the bone develops, which causes a deterioration in blood circulation in the bone tissue, which results in poorer healing. Micromovements in a fracture area load the screw-plate system, which results in a situation in which the screw can loosen.

These problems could be reduced by newer systems by fixing the screw in the plate with a spring between the plate and bone tissue, without reducing stability.

This new system, however, has the drawback that it has practical shortcomings in the wound, and that it is not as simple to handle, since at least two screws are often required to fasten the elements to each other. It is difficult with this system to simply and safely tighten the screws at a desired angular position relative to the implant plate, especially if the implant plate is thin, in order to include an important bone fragment in osteosynthesis and ensure anchoring in the implant plate.

Published U.S. Application No. 2005/0043736 A1 and U.S. Pat. No. 6,235,033 can be mentioned as an example. However, the inventions described in these references have certain limitations in terms of the necessary thickness of the implant plate and do not meet all the requirements now imposed on simple handling, precision, step-less flexibility in terms of angular position of the screw and safety during operations, where screws will fasten a bone fragment in different angular positions in the implant with varying thicknesses in narrow and often poorly accessible locations.

### BRIEF SUMMARY OF THE INVENTIVE IDEA

The present invention has the goal of eliminating the aforementioned problems and drawbacks during joining and fixing of two elements to each other, for example, affixing an implant to bone tissue with a screw, regardless of the screw's axial extent or angular position, and regardless of the implant's thickness.

According to the idea of the invention, the aforementioned problem is solved in that the mentioned first element, the implant, has a spherical cavity, in which a ductile elastic sleeve is arranged in the form of an annular sleeve, which is divided into segments, flanges, and which has a spherical shape that is complementary to the shape of the hole, and the sleeve has a conical threaded inside shape, which is complementary to the outer threaded shape of the screw head, in which the screw, when screwed into the bone tissue, can fasten the implant at any desired angle.

The invention consists of three parts which, when assembled, produce a stable implant combination, which comprises an implant plate with a through hole, a complementary sleeve for positioning in this hole and a screw that can be introduced to it. The implant plate is provided with a continuous spherical hole, in which the inlet opening for the hole is somewhat smaller than the outlet opening. The sleeve, whose outer surface is divided into flanges held together by a ring-shaped part in the end of the sleeve, is placed in this hole. The flanges are elastically bendable relative to the center of the sleeve, which means that it can easily be pressed into the spherical cavity of the plate, where it then stays by itself. The flanges of the sleeve delimit an inner conical cavity and each flange is provided on the inside with part of a conical thread. These thread parts together form a conical inside thread. The screw has a coarse thread for fastening in the bone and a cross-socket arranged in its head for a screwdriver, in order to be able to maneuver the bone screw continuously. The bone screw also has a self-tapping tip, above which there is a flat part to fit into the sleeve, in order to control it, when the bone screw is tightened further. The head of the bone screw is shaped conical with an outer cone-shaped thread, which, in the final stage of tightening, engages in the inner cone-shaped thread of the flanges of the sleeve. Beforehand, the screwdriver with connected bone screw can be directed at any angle and rotation, to be optimally introduced into the bone that is being fastened for osteosynthesis. With the self-tapping tip, the bone screw is introduced further, until the threaded cone of the bone screw in the head engages in the thread of the flanges of the sleeve. With the pressure that is exerted against the screw, it presses the sleeve down into the implant plate's lower hole, where it is wedged and therefore blocks the sleeve's capability of rotating when the bone screw is driven to a stop, when the flanges of the sleeve are firmly pressed into the spherical hole of the implant plate.

### BRIEF DESCRIPTION OF THE ACCOMPANYING DRAWINGS

FIG. 1 shows, in an arrangement according to the present invention, the implant plate, preferably made of metal, viewed from above in a horizontal view.

FIG. 2 shows the plate in FIG. 1, seen from the side in a vertical view, in a cross-section along A-A in FIG. 1.

FIG. 3 shows the implant plate in FIGS. 1 and 2, viewed in a perspective view, cut from above.

FIG. 4 shows, in an arrangement according to the present invention, the spherical, ductile and slotted sleeve, seen from the side in a perspective view.

FIG. 5 shows the spherical, slotted sleeve in FIG. 3, viewed from below.

FIG. 6 shows the spherical sleeve, viewed from the side.

FIG. 7 shows the spherical sleeve, viewed from above.

FIG. 8 shows the spherical sleeve, viewed from the side, in cross-section in a vertical view.

FIG. 9 shows a bone screw, seen from the tip.

FIG. 10 shows the bone screw, seen from above in a perspective view, with a screw head with a conical shell in an arrangement according to the present invention, viewed from the side.

FIG. 11 shows the bone screw in FIGS. 9 and 10, viewed from the side.

FIG. 12 shows the bone screw in the figure seen from above.

The annular sleeve, plate implant and screw in the screwed position, with the screw and sleeve viewed in a perspective view from the front, and with the plate 1 in a cross-section, are shown in FIG. 13 in relation to each other when they are assembled to a stable joint.

FIG. 14 shows the arrangement for holding and tightening of the screw.

### DETAILED DESCRIPTION OF A PREFERRED VARIANT OF THE INVENTION

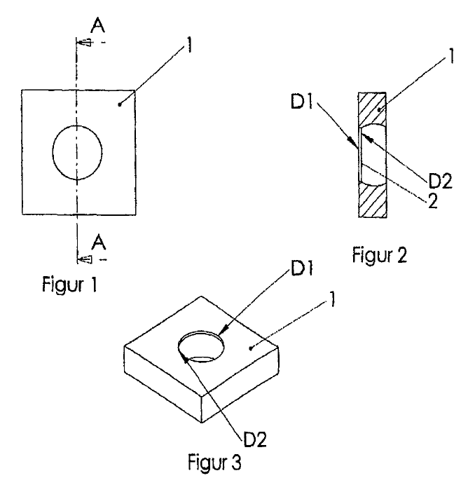

FIGS. 1 and 2 show an implant plate 1 in an arrangement according to the present invention, referred to as a plate below, made of a hard, machinable material, preferably metal. The plate can have varying thickness—for example, it can vary from about 2 mm and up. The plate 1 has a spherical through hole 2, which is drilled in plate 1, so that the center of the sphere of hole 2, seen from above, is shifted downward and therefore asymmetrically positioned in the plate, so that the upper hole opening of hole 2 has a diameter D1 that is less than the diameter D2 of the lower hole opening of hole 2.

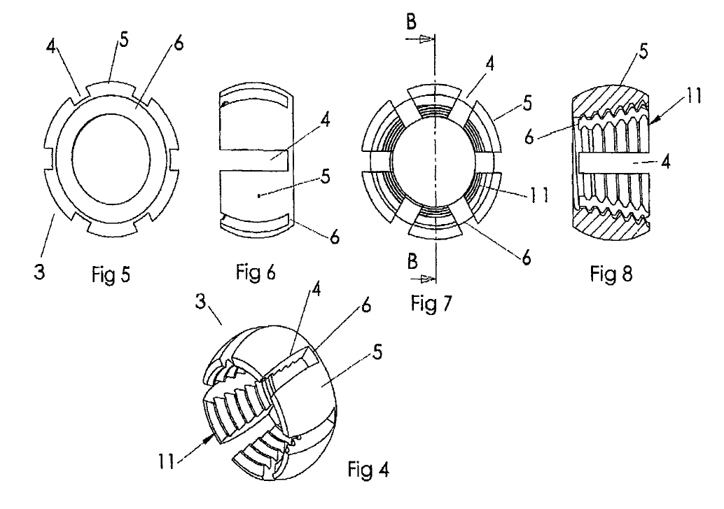

FIGS. 3, 4, 5, 6, 7 and 8 show a sleeve 3, made of a suitable material, preferably also metal, spherical in shape with flanges 5 bounded by slotted groove 4, with a remaining thin connection 6 between the flanges 5. This connection, in the form of a ring 6, permits the flanges 5 to be elastically deformed, so that the sleeve 3 can be clamped into the spherical hole 2 of plate 1. The sleeve 3 is held in place within the spherical hole 2, when the flanges 5 recover their normal shape. The spherical shape of sleeve 3 means that it can take up sloping positions without limitation. The inside of sleeve 3 is largely conical in shape and becomes cylindrical in its lower part with a screw of adapted dimensions, so that the sleeve is secured in the plate with the ring-shaped part 6 in the lower part of hole 2, preferably right outside of the lower hole opening D2 of the plate's spherical hole 2, which is larger than the upper hole opening D1. The inside of the conical part of sleeve 3 is provided with thread 11, and the flanges 5 have sharp edges to increase the holding effect.

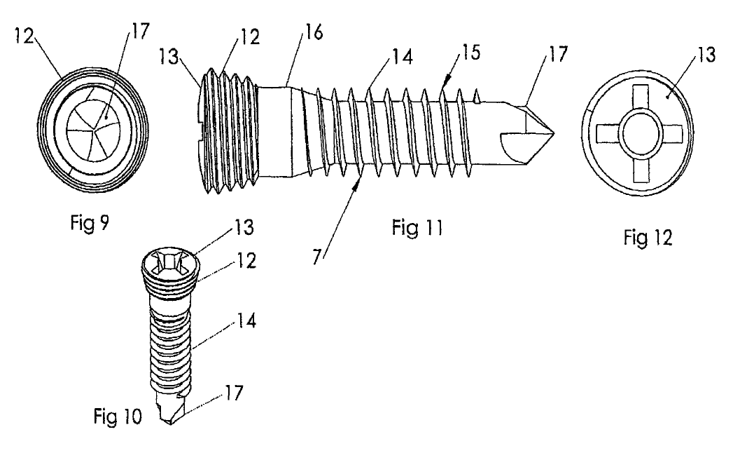

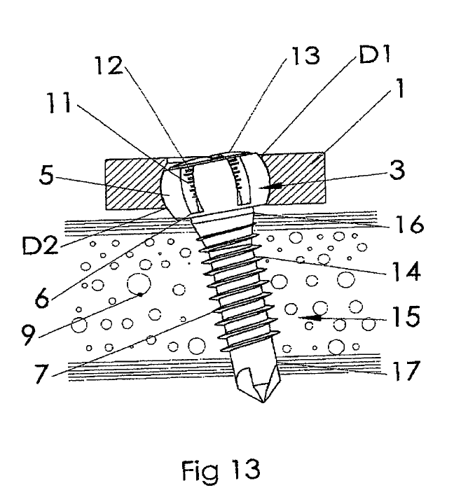

FIG. 8 shows the spherical sleeve 3, seen from the side in cross-section in a vertical view. During tightening of the screw 7, the threads 12 of the screw head engage with the threads 11 of the sleeve 3 at the same time that the slotted sleeve 3 expands and locks the sleeve against the sphere of hole 2 in the bottom of plate 1, since the outlet opening of the hole is somewhat larger than the inlet opening, so that the flanges are pressed against the edges of the sphere and block the tendency of the sleeve to rotate during tightening of the screw.

FIGS. 9, 10, 11 and 12 show screw 7 with its conical head 8 with threads 12 with a small pitch, relative to the threads in the lower part of the screw 7, which have a somewhat larger thread with greater pitch for suitable fixation in bone tissue.

FIG. 13 shows the sleeve 3, plate implant 1 and screw 7 in the tightened position, with the screw and sleeve seen in a perspective view from the front, and with the plate 1 and bone tissue 9 in cross section. The head 13 of screw I is entirely screwed into sleeve 3 and flanges 5 of sleeve 3 fill-up the spherical hole 2. The sleeve 3 preferably has a somewhat greater depth than the depth of spherical hole 2 in plate 1, in which the annular part 6 of sleeve 3, after introduction fully or partially, protrudes beneath the roughened lower edge surface of spherical hole 2 at D2 and, in this way, blocks the annular sleeve 3 from rotating with screw 7 during tightening.

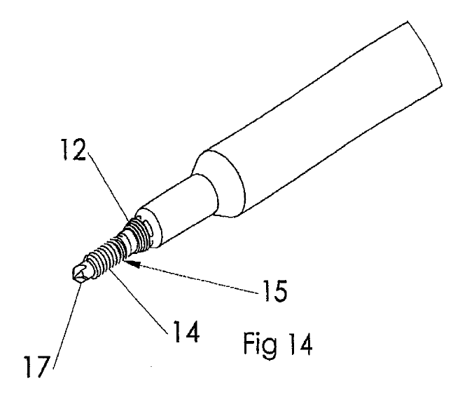

FIG. 14 shows an arrangement for holding a screw and tightening it.

When the two elements are to be assembled to each other with an arrangement according to the invention, for fastening the first element 1, the implant, in the form of a plate 1, to the other element 9, the bone tissue, by means of a screw 7, the screw 7 is pushed through the spherical hole 2, in which at least the flanges 5 on the annular sleeve 3 are situated, and through the ring-shaped part of sleeve 3, which is preferably situated outside of the lower edge of hole 2, the pressure on the screw forcing the flanges 5 of sleeve 3 against the protruding part of the spherical hole 2 of plate 1, which is roughened to increase the friction coefficient on it, and, in this way, the rotational tendency of the sleeve is blocked. Screw 7 is forced in, until it brushes against the other element, the bone tissue 9, whereupon the direction on screw 7 that is desired is determined and tightening of the lower thread 14 of screw 7 into the other element 9, the bone tissue, is continued. Final tightening occurs when the thread 12 of the screw stem 8 completely fills-up the conical thread 11 in sleeve 3 and therefore forces the sleeve 3 with its flanges 5 into the spherical hole 2 and finally the ring-shaped part 6 of sleeve 3 from the lower opening D2 of hole 2.

The present invention can naturally be modified within the scope of the accompanying patent claims without changing the scope of protection.

I claim:

1. An arrangement for fastening an implant against bone tissue with screw joint during surgery comprising; a screw having a head narrowing toward a tip of the screw, said head having an outside thread with a small pitch insertable into the implant, and having a main thread on a stem of the screw having a greater pitch than the small pitch of the screw head, the main thread being insertable into the bone tissue, the implant having an hole extending therethrough with a generally spherical wall, a ductile sleeve divided into segments which form a spherical shape generally matching the spherical shape of the wall of the opening in the implant, said sleeve having a threaded inside surface, which is complementary to the outside thread of the screw head, whereby the screw during tightening fastens to the implant at a variety of desired angles relative to the bone tissue and fixes the screw joint in a desired position.

2. An arrangement according to claim 1 wherein the sleeve includes a thin annular part that connects the segments.

3. An arrangement according to claims 2 wherein hole extending through the implant defines and upper opening and a lower opening, the spherical wall of the implant is positioned so that an upper opening is smaller than the lower opening.

4. An arrangement according to claim 3 wherein edges of the lower opening are roughened.

5. An arrangement according to claim 4 wherein the segments of the sleeve have a depth exceeding a depth of the hole in the implant so that the thin annular part of sleeve protrudes downward and below the lower roughened edge surface of the hole in a way that restricts rotation of sleeve when it is placed in the hole, sharp edges on the segment of the pressing against the spherical walls of the hole.

6. An arrangement according to claim 1 wherein the top of the conical head of the screw is arranged with formations that allow use of a screwdriver and an unthreaded part is disposed between the thread of the screw head and the lower thread of screw, the screw head during tightening expanding radially the spherical sleeve segments until the segments are anchored into engagement with the spherical walls of the hole and with the annular part of the sleeve is at least partially situated beneath the roughened lower edge surface of the spherical hole.

7. A method for fastening an implant relative to bone tissue by an arrangement according to claim 1, comprising: introducing the screw downward into the implant through the spherical cavity of the plate and throught the spherical sleeve in a direction and at an angular position relative to the bone tissue, causing the main thread of the screw to engage the bone tissue, tightening the screw until the thread of the screw head encounter the threaded inside surface of the sleeve, whereby final tightening occurs quickly due to the small pitch of the thread on the screw head and firmly locks the implant relative to the bone tissue, keeping the uppermost part of the screw at approximately the level of the implant.
